(19) United States
(12) Patent Application Publication
(10) Pub. No.: US 2003/0034031 A1
(43) Pub. Date: Feb. 20, 2003

(54) Pacifier and method of use thereof

(75) Inventors: Nimrod Lev, Ganei Yehuda (IL); Tal Pechter, Tel Aviv (IL)

Correspondence Address: DR. MARK FRIEDMAN LTD., Bill Polkinghorn, Discovery Dispatch, 9003 Florin Way, Upper Marlboro, MD, 20772, US

(73) Assignee: Sleep Up Ltd.

(21) Appl. No.: 10/270,507

(22) Filed: Oct. 16, 2002

Related U.S. Application Data
(63) Continuation-in-part of application No. 10/103,915, filed on Mar. 25, 2002.
Application No. 10/103,915 is a continuation-in-part of application No. 09/576,872, filed on May 22, 2000, now Pat. No. 6,371,112.

(51) Int. Cl. A61M 15/00

(52) U.S. Cl. 128/200.24; 128/201.26

(57) ABSTRACT

An improved pacifier and method of use thereof. The pacifier includes an artificial nipple to stimulate sucking when inserted in a mouth of a patient. The nipple is hollow and includes an interior volume serves as a channel of fluid communication between a first aperture and a second aperture. The first aperture is open to the interior volume of the nipple and the mouth. The second aperture is open to the interior volume of the nipple and an outside environment. The pacifier further includes an air flow valve with a first operational state in which the channel of fluid communication is closed and a second operational state in which the channel of fluid communication is open. The pacifier is constructed so that sucking causes the air flow valve to assume the first operational state while cessation of sucking allows the air flow valve to assume the second operational state. The method includes providing the described pacifier and regulating a flow of air in the channel of fluid communication by means of the air flow.

This application is a continuation in part of U.S. patent application Ser. No. 10/103,915 filed on Mar. 25, 2002 which was a continuation in part of U.S. patent application Ser. No. 09/576,872, filed on May 22, 200 which issued as U.S. Pat. No. 6,371,112 on Apr. 16, 2002.

### FIELD AND BACKGROUND OF THE INVENTION

The present invention relates to an improved pacifier and method of use thereof, which serve to facilitate mouth breathing while preventing collapse of the upper airway in infants and young children. More particularly, the present invention relates to treatment of breathing disorders including, but not limited to, apnea of infancy (AOI), sudden infant death syndrome (SIDS) and Rhinitis.

Currently accepted treatment for AOI typically includes continuous positive airway pressure (CPAP). CPAP, as currently practiced, involves connection of a pressurized air-delivering device to the mouth or nose of the patient. This device typically is connected to a pressurized air source in the form of a compressor or tank with a regulator. These pressurized air supplies are expensive, large, and noisy.

Delivery of a constant flow of a breathable gas mixture through the device maintains a constant positive pressure in the upper airway. This constantly applied pressure prevents the collapse of the airway described hereinabove (C. E. Sullivan et al. (1981) Lancet 1:862-865; M. H. Sanders et al. (1983) Chest 83:144-145). CPAP is effective because it prevents collapse of the airway and associated tiredness, fatigue, diminished intellectual function, and snoring and can even lengthen patient life expectancy (J. Hender et al. (1995) Eur. Respir. J. 8:222-229; C. Jenkinson et al. (1999) Lancet 353: 2100-2105).

However, patient compliance with CPAP treatment regimens is typically poor despite the proven efficacy of the treatment. Research suggests that 60 to 70% compliance is the norm and that the average patient use of CPAP is limited to 5 hours per night (N. P. Kribbs et al. (1993) Am. Rev. Respir. Dis. 147:887-895; H. Rauscher et al. (1993) Chest 103:1675-1680). In addition, many patients never begin treatment at all owing to the high cost of CPAP equipment. Further, infants and children are less likely to comply than adults because they are incapable of understanding the need for treatment. Parents of these young patients are often disturbed by the “medical” appearance of conventional CPAP equipment and find it embarrassing to show to relatives and caregivers.

Further, sleep apnea in infants and young children is often treated by surgical intervention. Such intervention necessarily includes all of the risks associated with anesthesia and surgery.

Infants are typically obligatory nasal breathers (Shannon D C In: Disorders of the Respiratory Tract In Children—SIDS and Apnea in infancy Kendig & Chernick eds (1990) W. B. Saunders Company, Philadelphia, Pa., USA pp 939-952.). Any change in airflows in the upper airways increase vulnerability to inadequate ventilation because: (1). the hypopharynx is shallow; (2) the tongue and epiglottis are more cephaled and (3) the mandible is more mobile (Tonkin S. (1975) Pediatrics 55:650-654). Normal infants typically have difficulty responding to nasal occlusion. Studies show that 44% of 6-week-old infants struggled but failed to establish an oral airway when the nostrils were pinched for 25 seconds (Swift P G F et al (1973) Arch Dis Child 48:947-950). The physiologic basis for the failure to maintain adequate oral ventilation during occlusion in both normal and at risk infants is unknown. This mechanism has been suggested as a cause of AOI which can lead to SIDS (Anderson R B et al (1971) Biol Neonate 18:395-398.

Thus, infants and young children suffering from excess nasal secretion tend to reject a pacifier during sleep in an effort to establish an alternate airway. This rejection may lead to sleep interruption when the comfort of sucking is subsequently missed.

There is thus a widely recognized need for, and it would be highly advantageous to have a device, and method, which facilitates mouth breathing in infants and young children devoid of the above limitations.

### SUMMARY OF THE INVENTION

According to one aspect of the present invention there is provided an improved pacifier. The pacifier includes: (a) an artificial nipple designed and constructed to stimulate sucking when inserted in a mouth of a young patient, the artificial nipple having a proximal portion insertable in the mouth and a distal portion protruding therefrom, the artificial nipple being hollow and includes an interior volume serving as a first channel of fluid communication between at least one first aperture and at least one second aperture; (b) the at least one first aperture forming a second channel of fluid communication between the interior volume of the artificial nipple and the mouth of the young patient; (c) the at least one second aperture forming a third channel of fluid communication between the interior volume of the artificial nipple and an outside environment; and (d) an air flow valve having a first operational state in which the first channel of fluid communication is closed and a second operational state on which the first channel of fluid communication is open. The pacifier is constructed so that sucking causes the air flow valve to assume the first operational state while cessation of sucking allows the air flow valve to assume the second operational state.

According to another aspect of the present invention there is provided a method of facilitating mouth breathing in a young patient. The method includes: (a) providing an artificial nipple designed and constructed to stimulate sucking when inserted in a mouth of a young patient, the artificial nipple having a proximal portion insertable in the mouth and a distal portion protruding therefrom, the artificial nipple being hollow and includes an interior volume, the artificial nipple further includes at least one first aperture and at least one second aperture so that a channel of fluid communication exists between an outside environment and the mouth of the young patient, the channel of fluid communication including at least a portion of the interior volume and (b) regulating a flow of air in the channel of fluid communication by means of an air flow valve having a first operational state in which the channel of fluid communication is closed and a second operational state on which the channel of fluid communication is open.

According to the method, sucking causes the air flow valve to assume the first operational state while cessation of sucking allows the air flow valve to assume the second operational state. Thus, it will be appreciated that the phrase “young patient”, as used herein and in the accompanying claims, refers to a patient that habitually uses a pacifier as a means of relaxation. This habitual pacifier use may occur, for example, during sleep.

According to further features in preferred embodiments of the invention described below, the improved pacifier further includes a retention guard designed and constructed to prevent swallowing of the artificial nipple. The retention guard may be, for example, attached to or integrally formed with the distal portion of the artificial nipple

According to still further features in the described preferred embodiments the improved pacifier further includes a filter designed and constructed to prevent the entry of foreign bodies thereto. The filter may be located, for example, in proximity to the at least one second aperture.

According to still further features in the described preferred embodiments the method further includes employing a retention guard designed and constructed to prevent swallowing of the artificial nipple, the retention guard being attached to or integrally formed with the distal portion of the artificial nipple

According to still further features in the described preferred embodiments the method further includes preventing the entry of foreign bodies into the channel of fluid communication by means of a filter located in proximity to the at least one second aperture.

The present invention successfully addresses the shortcomings of the presently known configurations by providing an improved pacifier and method of use thereof which facilitate mouth breathing while preventing collapse of the upper airway in infants and young children. The present invention does not require the use of conventional CPAP technology and provides a safe alternative to surgical intervention.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1a, 1b, 4a, 4b, 5a and 5b are cross sectional views of various embodiments of an improved pacifier according to the present invention;

FIGS. 2a and 2b are cross sectional views as in 1a and 1b showing how the improved pacifier is switched from one operational state to another during sucking; and

FIG. 3 is a simplified flow diagram of a method according to the present invention.

### DESCRIPTION OF THE PREFERRED EMBODIMENTS

The present invention is of an improved pacifier, and method of use thereof, which can be advantageously employed to facilitate mouth breathing while preventing collapse of the upper airway in infants and young children.

Specifically, the present invention can be used to treat breathing disorders including, but not limited to, apnea of infancy (AOI), sudden infant death syndrome (SIDS) and Rhinitis. Treatment, as used herein, is typically palliative or prophylactic treatment. Because the present invention is both safe and non-invasive, it may be used by either identified high-risk patients or by the general population of children that use conventional pacifiers.

The principles and operation of an improved pacifier according to the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

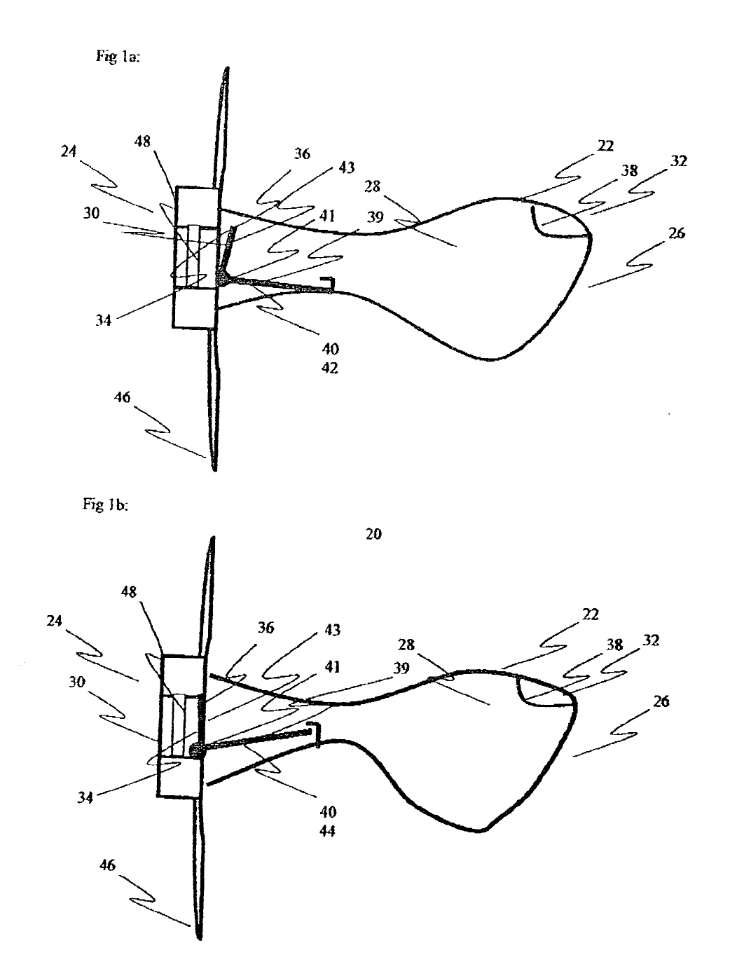

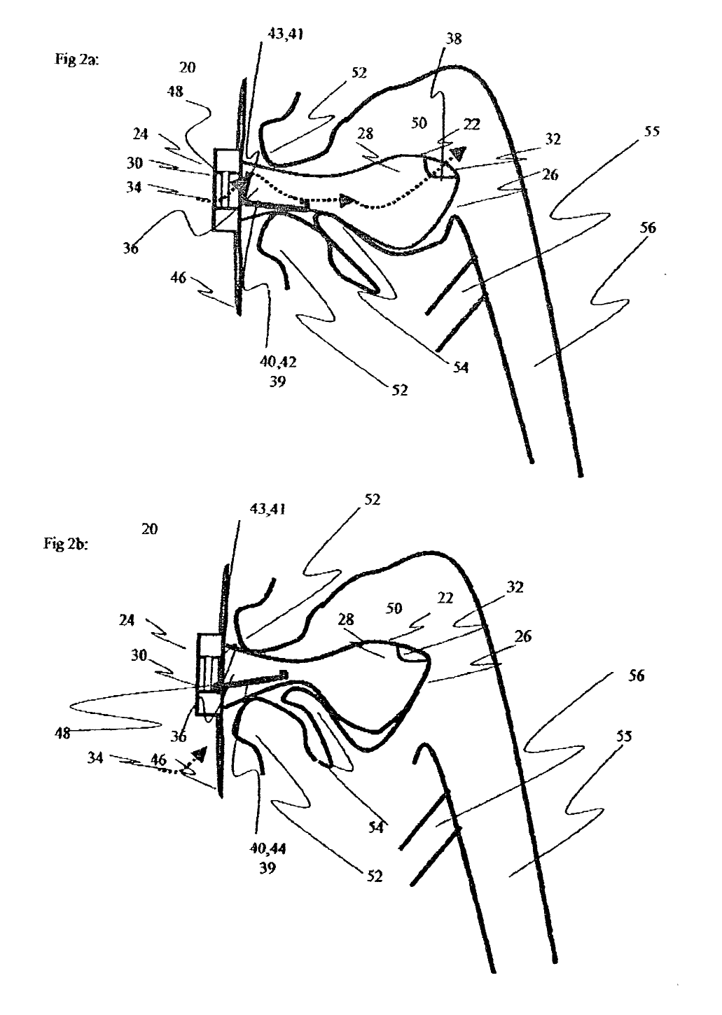

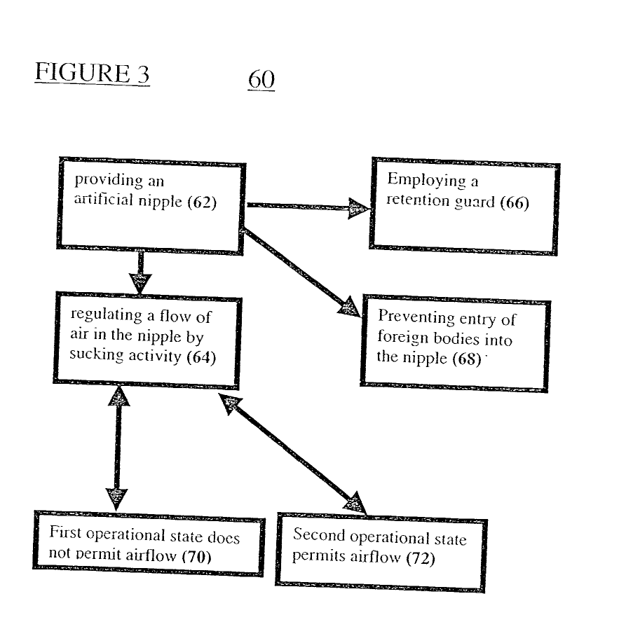

Referring now to the drawings, FIGS. 1a, 1b, 2a, 2b, 4a, 4b, 5a and 5b illustrate the improved pacifier 20 of the present invention either alone (FIGS. 1a, 1b, 4a, 4b, 5a and 5b) or in the context of a mouth of a patient engaged in sucking activity (FIGS. 2a and 2b). Pacifier 20 includes an artificial nipple 22 designed and constructed to stimulate sucking when inserted in a mouth 50 of a young patient. Nipple 22 is typically constructed of a soft plastic, rubber or silicon material as generally employed in conventional pacifiers although other materials may be employed without significantly affecting the functionality of the pacifier. Nipple 22 includes a proximal portion 26 insertable in mouth 50 of the patient. Nipple 22 further includes a distal portion 24 which protrudes from mouth 50 during use. Nipple 22 is hollow and includes an interior volume 28 which serves as a first channel 34 of fluid communication between at least one first aperture 32 and at least one second aperture 30. Second aperture 30 located in distal portion 24 of nipple 22 forms a second channel 36 of fluid communication between interior volume 28 of artificial nipple 22 and an external environment. First aperture 32 forms third channel 38 of fluid communication between the interior volume 28 of artificial nipple 22 and mouth 50. Pacifier 20 further includes an air flow valve 40 having a first operational state 44 (FIGS. 1b, 2b, 4b and 5b) in which the first channel 30 of fluid communication is closed and a second operational state 42 (FIGS. 1a, 2a, 4a and 5a) in which the first channel 34 of fluid communication is open. Pacifier 20 is constructed so that extension of tongue 54 in an upward direction against nipple 22 during sucking causes air flow valve 40 to assume first operational state 44. Subsequent cessation of sucking allows the air flow valve 40 to assume second operational state 42 as tongue 54 moves downward from nipple 22.

Pacifier 20 preferably further includes a retention guard 46 designed and constructed to prevent swallowing of artificial nipple 22. Retention guard 46 may be, for example, attached to or integrally formed with the distal portion 24 of artificial nipple 22 so that it naturally resides outside of lips 52 during use. Pacifier 20 may further include a filter 48 designed and constructed to prevent the entry of foreign bodies thereto. Filter 48 may be located, for example, in proximity to second aperture 30.

It will be appreciated that different configurations of valve 40 give rise to different embodiments of the claimed invention. Several such configurations are offered as non-limiting examples.

FIGS. 1a, 1b, 2a and 2b illustrate a valve 40 including a lever 39 connected via an axle 41 to a flap 41. Pressure of tongue 54 on nipple 22 raises lever 39 causing axle 41, mounted on a surface of interior volume 28 of nipple 22 to rotate thereby causing flap 41 to close second aperture 30. A similar valve could be employed to close first aperture 32 without significantly changing the function of the invention.

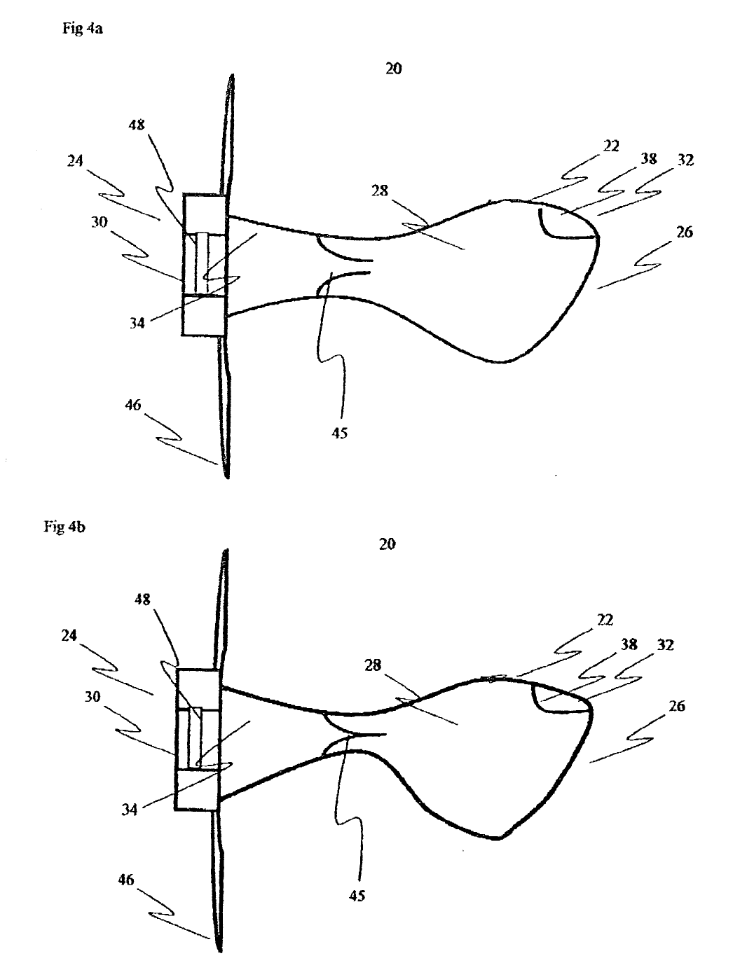

FIGS. 4a and 4b illustrate a valve 40 including two flexible leaves 45. Pressure of tongue 54 on nipple 22 causes leaves 45 to come into contact one with the other thereby closing first channel 34.

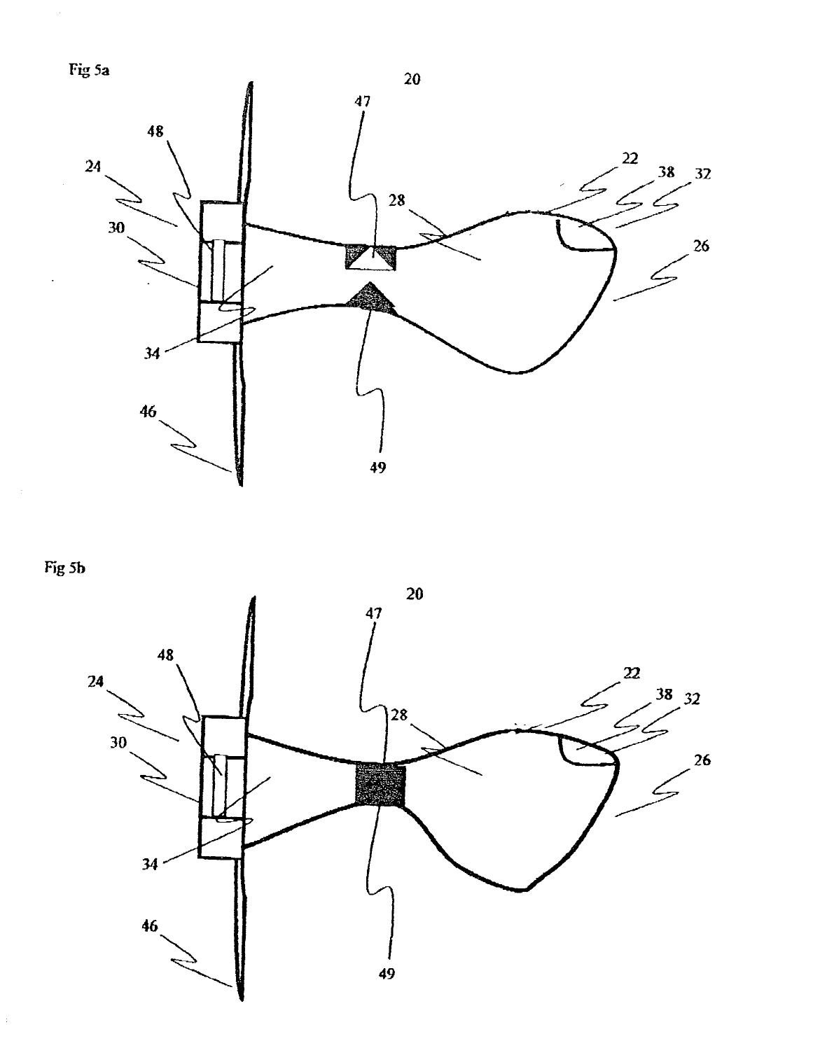

FIGS. 5a and 4b illustrate a valve 40 including a convex portion 49 and a concave portion 47. Pressure of tongue 54 on nipple 22 causes convex portion 49 to come into contact with concave portion 47 thereby closing first channel 34.

Leaves 45 and convex portion 49 and concave portion 47 may be integrally formed with or attached to a surface of interior volume 28 of nipple 22. These parts of valve 40 may be constructed, for example, of the same rubber or silicon used to construct nipple 22.

The present invention is further embodied by a method 60 of facilitating mouth breathing in a young patient. Method 60 includes providing 62 artificial nipple 22 designed and constructed to stimulate sucking when inserted in mouth 50 of the young patient. Nipple 22 has a proximal portion 26 insertable in mouth 50 and a distal portion 24 protruding therefrom. Nipple 22 is hollow and includes an interior volume 28 with first aperture 32 and second aperture 30 connected by channel of fluid communication 34 as described hereinabove. Method 60 further includes regulating 64 a flow of air in channel of fluid communication 34 by means of air flow valve 40. As described hereinabove, valve 40 has a first operational state 44 in which channel of fluid communication 34 is closed and second operational state 42 in which channel of fluid communication 34 is open.

As described hereinabove sucking 70 causes air flow valve 40 to assume first operational 44 state while cessation 72 of sucking allows the air flow valve to assume the second operational state. Thus, it will be appreciated that the phrase “young patient”, as used herein and in the accompanying claims, refers to a patient that habitually uses a pacifier as a means of relaxation. This habitual pacifier use may occur, for example, during sleep.

Preferably, method 60 further includes employing 66 retention guard 46 designed and constructed to prevent swallowing of artificial nipple 22. Retention guard 46 may be, for example, attached to or integrally formed with distal portion 24 of artificial nipple 22.

Optionally, but also preferably, method 60 further includes preventing 68 the entry of foreign bodies into channel 36 or 34 of fluid communication by means of a filter located in proximity to second aperture 30.

In summary, improved pacifier 20 of the present invention allows air to enter mouth 50 via interior volume 28 of nipple 22 and be directed to airway 56 during inspiration. Pacifier 20 further serves to prevent entry of air into esophagus 55 during sucking. This unique action both helps to establish oral access to the airway to facilitate mouth breathing and reduces frustration of a user with an occluded nasal passage. Thus a primary benefit of the invention is a prophylactic or palliative treatment for disorders such as AOI or SIDS while a secondary benefit of the invention is a reduction in frequency of waking for young patients with an occluded nasal passage.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. An improved pacifier, the pacifier comprising:
(a) an artificial nipple designed and constructed to stimulate sucking when inserted in a mouth of a young patient, said artificial nipple having a proximal portion insertable in said mouth and a distal portion protruding therefrom, said artificial nipple being hollow and comprising an interior volume serving as a first channel of fluid communication between at least one first aperture and at least one second aperture;
(b) said at least one first aperture forming a second channel of fluid communication between said interior volume of said artificial nipple and said mouth of said young patient;
(c) said at least one second aperture forming a third channel of fluid communication between said interior volume of said artificial nipple and an outside environment; and
(d) an air flow valve having a first operational state in which said first channel of fluid communication is closed and a second operational state on which said first channel of fluid communication is open;
wherein sucking causes said air flow valve to assume said first operational state while cessation of sucking allows said air flow valve to assume said second operational state.

2. The improved pacifier of claim 1, further comprising a retention guard designed and constructed to prevent swallowing of said artificial nipple, said retention guard being attached to or integrally formed with said distal portion of said artificial nipple

3. The improved pacifier of claim 1, further comprising a filter designed and constructed to prevent the entry of foreign bodies thereto, said filter located in proximity to said at least one second aperture.

4. A method of facilitating mouth breathing in a young patient, the method comprising:
(a) providing an artificial nipple designed and constructed to stimulate sucking when inserted in a mouth of a young patient, said artificial nipple having a proximal portion insertable in said mouth and a distal portion protruding therefrom, said artificial nipple being hollow and comprising an interior volume, said artificial nipple further comprising at least one first aperture and at least one second aperture so that a channel of fluid communication exists between an outside environment and said mouth of said young patient, said channel of fluid communication including at least a portion of said interior volume;
(b) regulating a flow of air in said channel of fluid communication by means of an air flow valve having a first operational state in which said channel of fluid communication is closed and a second operational state on which said channel of fluid communication is open;
wherein sucking causes said air flow valve to assume said first operational state while cessation of sucking allows said air flow valve to assume said second operational state.

5. The method of claim 4, further comprising employing a retention guard designed and constructed to prevent swallowing of said artificial nipple, said retention guard being attached to or integrally formed with said distal portion of said artificial nipple

6. The method of claim 4, further comprising preventing the entry of foreign bodies into said channel of fluid communication by means of a filter located in proximity to said at least one second aperture.
